(19) United States
(12) Patent Application Publication
(10) Pub. No.: US 2005/0152857 A1
(43) Pub. Date: Jul. 14, 2005

(54) Method and preparation for reducing sunburn cell formation in skin

(75) Inventors: Joseph C. Dinardo, Raleigh, NC (US); Joseph A. Lewis II, Chesterfield, VA (US); Birgit Neudecker, Detmold (DE); Falko Diedrich, Detmold (DE); Eberhard Wieland, Stuttgart (DE)

Correspondence Address: DAVIDSON, DAVIDSON & KAPPEL, LLC, 485 SEVENTH AVENUE, 14TH FLOOR, NEW YORK, NY, 10018, US

(73) Assignee: PCR Technology Holdings, LC, Richmond, VA (US)

(21) Appl. No.: 10/825,265

(22) Filed: Apr. 15, 2004

Related U.S. Application Data
(60) Provisional application No. 60/535,024, filed on Jan. 8, 2004.

(51) Int. Cl. A61K 7/42

(52) U.S. Cl. 424/59

(57) ABSTRACT

A method for reducing sunburn cells in human skin includes applying a topical preparation comprising an amount of an agent effective to reduce the formation of sunburn cells in human skin, and exposing the skin to ultraviolet radiation. The agent includes idebenone or a derivative of idebenone.

Priority is claimed to provisional application No. 60/535,024, filed on Jan. 8, 2004, and which is hereby incorporated by reference herein.

The present invention relates in general to the formation of sunburn cells in human skin exposed to ultraviolet radiation, such as sunlight, and to sun sensitivity, and relates in particular to a method for reducing the formation of sunburn cells in human skin and reducing an increase in sun sensitivity in human skin exposed to ultraviolet radiation.

### BACKGROUND

UV radiation generally encompasses light in the wavelength range of 200-400 nm, with UVA having a wavelength of about 320-440 nm, UVB a wavelength of 290-320 nm, and WVC a wavelength of less than about 280 nm. Acute UV radiation exposure, e.g., exposure to sunlight or man-made UV sources, is associated with formation of dyskeratotic cells (also called sunburn cells) in the epidermis. Sunburn cells are epidermal cells with an eosinophilic cytoplasm and either no nucleus or a contracted, irregular, nucleus, when stained with hematoxylin and eosin. The formation of sunburn cells is believed to indicate damage to cellular DNA by UV radiation, and in particular UVB radiation.

U.S. Pat. No. 6,132,737 describes the use of ascorbic acid-containing compositions in reducing sunburn cell formation in human skin.

Traditional sunscreen compositions containing titanium dioxide and chemical sunscreens do provide some degree of protection against formation of sunburn cells. However, these products still permit formation of significant numbers of sunburn cells in skin which is exposed to UV radiation.

Certain substances applied to the skin are known to cause an increase in sun sensitivity (sensitivity to UV light) when the skin is exposed to UV light. For example, an increase in the number of sunburn cells formed relative to untreated skin may occur. Such substances include alpha hydroxy acids (AHA), such as glycolic acid, lactic acid, malic acid, tartaric acid, citric acid, and the like; beta hydroxy acids (BHA), such as salicylic acid and the like; and retinoids, such as retinol, retinal, retinyl palmitate, retinoic acid, tazarotene—acetylenic retinoids, and other ester derivatives of Vitamin A and the like.

Traditional AHAs are widely used as ingredients in cosmetics. Several studies suggest that AHAs can increase the sensitivity of skin to UV light. One such study performed in order to determine whether short-term dermal treatment with glycolic acid, a representative AHA, can enhance the damaging effects of UV light is discussed in Kaidbey et al., “Topical glycolic acid enhances photodamage by ultraviolet light,” Photodermatology, Photoimmunology & Photomedicine, February 2003, 19(1), pp. 21-27.

The duration of the effect of AHAs on the sensitivity of skin to UV light was also examined. The backs of 29 Caucasian subjects were treated, once daily, 6 days per week with either 10% glycolic acid (pH 3.5) or placebo in a randomized double-blinded study. At the end of 4 weeks, sites within each treated area were exposed to 1.5 MED (minimal erythema dose) of UV light, determined on previously untreated skin. Short-term application of 10% glycolic acid was shown to cause enhanced sensitivity to UV light, including increased sunburn cell induction. This photosensitivity was reversed within a week of terminating treatments.

German patent document DE 3 049 039, European patent document 788 793, U.S. Pat. No. 4,436,753, U.S. Pat. No. 5,059,627, U.S. Pat. No. 5,916,925 and WIPO publication number 99 07 355 describe oral, parenteral or percutaneous preparations containing idebenone or its derivatives for the treatment of dementia, circulatory disturbances or for the induction of a neural growth factor. Japanese patent document 1 279 818 describes the use of idebenone and its derivatives in various preparations for coloring hair.

### SUMMARY OF THE INVENTION

The present invention provides a method for reducing the formation of sunburn cells in human skin. The method includes applying to the skin a topical preparation comprising an amount of an agent effective to reduce the formation of sunburn cells in human skin, and exposing the skin to ultraviolet radiation. The agent includes idebenone or a derivative of idebenone.

The ultraviolet radiation may be ultraviolet B radiation from sunlight or man-made UV radiation sources.

The idebenone or derivative of idebenone may have a concentration of from about 0.001% to about 30%, by weight of the preparation. The preparation may be in the form of a lotion, a cream, a gel, a solution, a spray, a cleanser, a powder, an ointment, a wax, a lipstick, a soap, a shampoo, or a hydroalcoholic solution.

The present invention also provides a method for preventing an increase in sun sensitivity in human skin in the presence of a compound capable of causing the increase in sun sensitivity. The method includes applying to the skin a topical preparation comprising an amount of an agent effective to reduce the formation of sunburn cells in human skin, and exposing the skin to ultraviolet radiation. The agent includes idebenone or a derivative of idebenone. The compound may be included in the topical preparation so that the compound and the agent are applied to the skin together. In other embodiments, the compound and the topical preparation may be applied to the skin separately.

Moreover, the present invention provides a method for reducing the formation of sunburn cells in human skin. The method includes making available for purchase a topical preparation directed to reducing the formation of sunburn cells in human skin. The topical preparation includes an amount of an agent effective to reduce the formation of sunburn cells in human skin. The agent includes idebenone or a derivative of idebenone.

In addition, the present invention provides a topical preparation for reducing the formation of sunburn cells in human skin. The preparation includes an ultraviolet filter substance and an amount of idebenone or a derivative of idebenone effective to reduce the formation of sunburn cells in human skin.

The present invention also provides a topical preparation for reducing the formation of sunburn cells in human skin. The preparation includes a compound capable of causing an increase in sun sensitivity in human skin and an amount of idebenone or a derivative of idebenone effective to reduce the formation of sunburn cells in the human skin.

It has been found in clinical testing that the method and preparation of the invention provided, for example, a 31% to 44% reduction in the number of sunburn cells by treating human skin with a idebenone-containing compositions (0.01 wt. %, 0.1 wt. %, and 1.0 wt. % idebenone, respectively) prior to exposure to UV radiation. Moreover, it has been found in clinical testing that including, for example, 0.5 wt. % idebenone in a composition containing glycolic acid prevented the increase in formation of sunburn cells in UV-exposed human skin that is commonly produced by such compositions.

### DETAILED DESCRIPTION

Idebenone (6-(10-hydroxydecyl)-2,3-dimethoxy-5-methyl-1,4-benzoquinone) is characterized by the following structural formula:

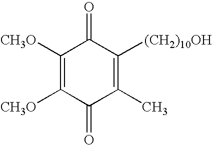

Chemical derivatives of idebenone may also be suitable for use in a method according to the present invention. Such derivatives may include, for example, esters and salts of idebenone, or protein bound forms, or other derivatives. Examples of idebenone derivatives include esters of idebenone where idebenone is esterified using glycosaminoglycans and/or their salts, for example hyaluronic acid having a molecular weight of 1 to 1,000,000 and its salts or hyaluronidase inhibitors, such as for example inter-alpha-trypsin inhibitor.

An example of a hydrophilic idebenone ester (separate synthesis) is idebenone sulphonic acid, characterized by the following structural formula:

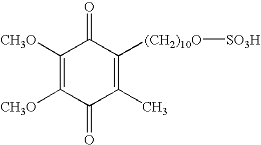

An exemplary synthesis of idebenone sulphonic acid was performed as follows: idebenone was reacted with pyridine-SO₃ and the reaction was then stopped using 1 N hydrochloric acid. After shaking out the organic phase using ethyl acetate, the organic phase was dried and concentrated under vacuum. The residue was dissolved in water and insoluble products centrifuged off. The hydrophilic idebenone ester thus recovered is suitable for application according to the invention in aqueous cosmetic and dermatological preparations.

Compositions according to the present invention may contain a concentration of idebenone or a derivative of idebenone of about 0.001-30%, 0.01-10.0%, 0.1-2.0%, or 0.5% to 1.0% by weight of the composition.

The compositions may be cosmetic, dermatologic, or pharmaceutical preparations or compositions, and may exist in a wide variety of forms, such as emulsions, suspensions, solutions and the like. In certain embodiments, the compositions are in the form of lotions, creams, and other types of cosmetic compositions.

For administration, the cosmetic and dermatological preparations of the invention may be applied to the skin in adequate quantity in the manner conventional for cosmetics.

Cosmetic and dermatological preparations of the invention may exist in various forms. Hence, they may be, for example a solution, an anhydrous preparation, an oil-free preparation, an emulsion or microemulsion of the type water-in-oil (W/O) or of the type oil-in-water (O/W), a multiple emulsion, for example of the type water-in-oil-in-water (W/O/W), a gel, a solid stick, an ointment or even an aerosol. It is also advantageous to administer idebenone and/or its derivatives in encapsulated form, for example in collagen matrices and other conventional encapsulation materials, for example as cellulose encapsulations, in gelatine, wax matrices or liposomally encapsulated.

It is also possible and advantageous within the scope of the present invention to add idebenone and/or its derivatives, such the sulphate of idebenone, for example, to aqueous systems or surfactant preparations for cleansing the skin.

The cosmetic and dermatological preparations of the invention may contain cosmetic auxiliaries, as are used conventionally in such preparations, for example preservatives, bactericides, perfumes, substances for preventing foaming, dyestuffs, pigments which have a coloring effect, thickening agents, surfactant substances, emulsifiers, softening, moisturizing and/or moisture-retaining substances, fats, oils, waxes or other conventional constituents of a cosmetic or dermatological formulation, such as alcohols, polyols, polymers, foam stabilizers, electrolytes, organic solvents or silicone derivatives.

In particular, idebenone and its derivatives may also be combined according to the invention with one or more traditional or other anti-oxidants and/or free-radical absorbers that are suitable or conventional for cosmetic and/or dermatological applications. Such anti-oxidants include, for example, one or more of the following: antioxidant enzymes (for example superoxide dismutase, catalase, glutathione peroxidase, glutathione S-transferase, glutathione reductase), antioxidant botanical extracts (for example green tea, white tea, black tea, licorice, grape, bilberry), plant growth factors (for example N-furfuryladenine), amino acids (for example glycine, histidine, tyrosine, tryptophan) and their derivatives, imidazoles (for example urocanic acid) and their derivatives, peptides, such as D,L-carnosine, D-carnosine, L-carnosine and their derivatives (for example anserine), carotinoids, carotenes (for example alpha-carotene, beta-carotene, lycopene) and their derivatives, chlorogenic acid and its derivatives, lipoic acid and its derivatives (for example dihydrolipoic acid), aurothioglucose, propylthiouracil and other thiols (for example thioredoxin, glutathione, cysteine, cystine, cystamine and their glycosyl, N-acetyl, methyl, ethyl, propyl, amyl, butyl and lauryl, palmitoyl, oleyl, gamma-linoleyl, cholesteryl and glyceryl esters) and their salts, dilauryl thiodipropionate, distearyl thiodipropionate, thiodipropionic acid and their derivatives (esters, ethers, peptides, lipids, nucleotides, nucleosides and salts) and sulphoximine compounds (for example buthionine sulphoximines, homocysteine sulphoximine, buthionine sulphones, pentathionine sulphoximine, hexathionine sulphoximine, heptathionine sulphoximine) in very low, acceptable doses (for example pmole to μmoles/kg), also (metal) chelating agents (for example alpha-hydroxy fatty acids, palmitic acid, phytic acid, lactoferrin), alpha-hydroxy acids (for example citric acid, lactic acid, malic acid, mandelic acid, gluconolactone, lactobionic Acid), humic acid, colic acid, colic extracts, bilirubin, biliverdin, EDTA, EGTA and their derivatives, unsaturated fatty acids and their derivatives (for example gamma-linolenic acid, linolic acid, oleic acid), folic acid and their derivatives, ubiquinone and ubiquinol and their derivatives, vitamin C and derivatives (for example ascorbyl palmitate, Mg-ascorbyl phosphate, ascorbyl acetate), tocopherols and derivatives (for example vitamin E acetate), vitamin A and derivatives (for example vitamin A palmitate) and coniferyl benzoate of benzoin resin, rutinic acid and their derivatives, butylhydroxy toluene, butylhydroxy anisole, nordihydroguaiacic acid, nordihydroguaiaretic acid, trihydroxybutyrophenone, uric acid and its derivatives, mannose and its derivatives, sesamol, sesamolin, zinc and its derivatives (for example ZnO, ZnSO₄), selenium and its derivatives (for example selenium methionine), stilbenes and their derivatives (for example stilbene oxide, trans-stilbene oxide) and suitable derivatives (salts, esters, ethers, sugars, nucleotides, nucleosides, peptides and lipids) of these said active ingredients.

The quantity of the aforementioned anti-oxidants (one or more compounds) in the preparations may be, for example, 0.0001 wt. % to 30 wt. %, 0.05 wt. % to 20 wt. %, or 1 to 10 wt. %, based on the total weight of the preparation.

Emulsions according to the invention may contain, for example the said fats, oils, waxes and other adipoids, and water and an emulsifier, as is used conventionally for such a type of formulation.

Also any mixtures of such oil and wax components can be used advantageously within the scope of the present invention. It may also optionally be advantageous to use waxes, for example cetyl palmitate, as the single lipid component of the oil phase.

Gels according to the invention may contain alcohols of low C number, for example ethanol, isopropanol, 1,2-propane diol, glycerine and water or an above-mentioned oil in the presence of a thickening agent,-which for oily-alcoholic gels is preferably silicon dioxide or an aluminium silicate, for aqueous-alcoholic or alcoholic gels is preferably a polyacrylate.

Conventional highly volatile, liquefied propellants, for example hydrocarbons (propane, butane, isobutane), which may be used alone or mixed with one another, are suitable as propellants for preparations which can be sprayed from aerosol containers according to the invention. Compressed air can also advantageously be used.

Preparations of the invention may also contain filter substances that absorb UV radiation, or sunscreens, wherein the total quantity of filter substances is, for example 0.1 wt. % to 30 wt. % or 0.5 wt. % to 10 wt. %, based on the total weight of the preparation. The preparations may also serve as sunscreen agents for the skin. Such UV filter substances include, for example, the following: avenobenzene, cinoxate, dioxybenzone, homosalate, menthyl anthranilate, octocrylene, octyl methoxycinnamate, octyl salicylate, oxybenzone, padimate O, phenylbenzimidazole sulfonic acid, sulisobenzone, titanium dioxide, trolamine salicylate, and zinc oxide.

A preparation according to the invention may be an oil and water, water and oil, a water and oil, or a water emulsion including, for example, by weight of the preparation:
from about 10% to about 90% of water;
from about 0% to about 20% of at least one humectant;
from about 0% to about 20% of at least one emollient;
from about 0% to about 20% of at least one ester;
from about 0% to about 10% of at least one thickener;
from about 0% to about 2% of at least one preservative;
from about 0% to about 1% of color; and
from about 0% to about 1% of fragrance.

A preparation according to the invention may be an oil and water, water and oil, a water and oil, or a water emulsion including, for example, by weight of the preparation:
from about 50% to about 90% of water;
from about 1% to about 10% of glycerin;
from about 1% to about 5% of cetyl ricinoleate;
from about 1% to about 5% of isohexadecane
from about 1% to about 5% of ceresin;
from about 0.5% to about 2.5% of sericin;
from about 0.1% to about 1% of glycosaminoglycans;
from about 0.1% to about 1% of dimethicone; and
from about 0.001% to about 30% of idebenone.

In the method according to the invention idebenone-containing compositions are applied to human, skin. The compositions may be applied once or more times per day depending on the activities the particular individual is engaged in. For example, an individual engaging in normal workday activities may wish to apply the compositions twice a day, once in the morning, and once in the evening, in conjunction with normal grooming. On the other hand, if the individual plans outdoor activities such as sunbathing and athletics, the compositions may be applied prior to, and during, such activities, much like a sunscreen composition is applied periodically during the day. The compositions may be used to reduce sunburn cell formation on the face and neck, by applying appropriate idebenone compositions to the face and neck areas. However, the idebenone compositions may also be applied to the entire body, particularly areas which are not covered by clothing, such as the arms, neck, and lower legs.

It has been found the application of idebenone-containing compositions in this manner significantly reduces the number of sunburn cells which are formed upon exposure to UV radiation, particularly UVB radiation. Referring to Example 2 below, generally, the extent of sunburn cell formation is determined by obtaining slide preparations of skin cells according to well known histological techniques. The slides are then stained with hematoxylin-eosin, and the number of dyskeratotic cells per high power field (generally 100× magnification) is counted. Generally a number of high power fields are counted, for example 25 to 100 high power fields per sample, to ensure relability of results. Mean sunburn cell counts from skin areas treated with the Idebenone-containing composition are compared with the sunburn cell counts from untreated skin and placebo treated skin are compared.

The idebenone used in the examples according to the present invention presented below was obtained from Alpin Chemical company, although idebenone is available from other sources.

The invention will be described in connection with the following examples which are set forth for the purposes of illustration only.

### EXAMPLE 1

Idebenone-containing Formulas 1, 2 and 3 were prepared as follows:

| | w/w % | | |
|---|---|---|---|
| | Formula: | | |
| | 1 | 2 | 3 |
| Idebenone | 1.0 | 0.10 | 0.01 |
| SD Alcohol 40B | 75.0 | 75.00 | 75.00 |
| DI Water | 24.0 | 24.90 | 24.09 |

To prepare Formulas 1, 2 and 3, the idebenone was added directly to the SD Alcohol 40B while stirring until dissolved. DI Water was then added to the solution with continued stirring.

### EXAMPLE 2

The idebenone-containing Formulas 1, 2, and 3 of Example 2 and a non-treated control (NT) were applied to human skin in a 2-week human sunburn cell assay study.

The panel composition was six different healthy adult volunteers between the ages of 18 and 53. All were in excellent health, and without any significant internal or dermatological diseases. The subjects were carefully screened to ensure they were not taking any medications. All had a clear back free of blemishes or a tan and were of skin types II and III according to the following classification scheme:
Type I: always burns easily, never tans (sensitive)
Type II: always burns easily; tans minimally (sensitive)
Type III: burns moderately; tans gradually—normal skin (light brown)

The subjects were given detailed instructions to avoid any direct exposure to sunlight and to minimize any incidental exposure to their backs for the entire duration of the study. No other topical products were allowed, and no medications including over-the-counter (OTC) products were to be used except for Acetaminophen (e.g., common Tylenol®) for the relief of transient pains or headaches.

Individual test sites were delineated over the midback region. The test products were randomly allocated amongst the test sites according to a randomization schedule prepared by the investigator. The subjects came in once daily to the laboratory and received supervised applications of the test products to the allocated test sites. All test products were dispensed by a technician using 1 cc disposable plastic tuberculin syringes. Each site received 100 μl (2 μl/square cm) of, as appropriate, Formula 1, 2 or 3 of Example 3, or no formula (normal, untreated skin site). The test product was then spread uniformly throughout the 5×10 cm rectangular test site using a finger cot. The subjects received once daily application for two consecutive weeks (except on weekends).

The light source used was a 150 watt xenon arc solar simulator equipped with a UV reflecting diachronic mirror and a 1 mm thick Schott WG-320 filter to produce simulation of the solar spectrum. A 1 mm thick UG5 filter was added to remove reflected heat and remaining visible radiation. Warm up time of the lamp before use was 20-25 minutes. Total irradiance at skin level was measured with a calibrated Eppley Thermopile and the UVB component was monitored with a UVB radiometer (International Light Inc, Newburyport, Mass.). The size of the irradiated field was approximately a 1 cm diameter circle.

Two days prior to the end of the study, the MED (minimal erythema dose) for each subject was determined by exposing several normal untreated skin sites over the midback area to a series of exposures in 25% dose increments from the solar simulator. The MED was defined as the time of exposure required to produce a minimally perceptible erythema 20±4 hours after exposure. Visual grading of the MED was done under standardized lighting conditions when the subjects returned to the testing facility approximately 24 hours after irradiation. The MED was recorded in the appropriate case record form.

Approximately ten minutes after the last topical application of the test products, a circular area measuring 1 cm in diameter within each treated test site was exposed once to a signal does of 1.5 MED's. The MED was based on the determination in the nearby normal skin site using the solar stimulator. 20±2 hours later, shave biopsy (approximately 4×4 mm) was obtained from each irradiated site following injection of a local anesthetic (xylocaine). The skin specimens were immediately fixed in 10% buffered formalin.

Histology: the fixed specimens were processed routinely, embedded in paraffin and then sectioned and stained with hematoxylin-eosin. The numbers of sun burn cells (SBC) were determined in at least 12 sections at 50 u intervals. A minimum of 70 High Power Fields (HPF) was counted from each biopsy and the average number of SBCs per HPF determined. All specimens were counted in a blinded manner by the investigator, where all slides were identified only by subject number and test code.

It has been found that the method of the invention provided between a 31% to a 44% reduction (depending on the concentration tested) in the number of sunburn cells formed when compared to untreated skin which is exposed to the same degree of UV radiation.

The mean number of sunburn cells (SBC) for each subject is set forth below in Table I.

TABLE I
Mean Number of SBC Per High Power Field

| Subject | | | | |
|---|---|---|---|---|
| Number | Formula 1 | Formula 2 | Formula 3 | NT Site |
| 1 | 0.33 | 0.46 | 0.29 | 0.69 |
| 2 | 0.32 | 0.36 | 0.29 | 0.19 |
| 3 | 0.39 | 0.30 | 0.24 | 0.33 |
| 4 | 0.70 | 1.05 | 1.43 | 2.05 |
| 5 | 0.20 | 0.17 | 0.13 | 0.21 |
| Mean | 0.39 | 0.47 | 0.48 | 0.69 |
| Delta % | 44.00 | 33.00 | 31.00 | N/A |

Formula 1 (1.00 wt. % idebenone)
Formula 2 (0.10 wt. % idebenone)
Formula 3 (0.01 wt. % idebenone)
NT Site - Normal untreated skin exposed to UV

Conclusion: Formulas 1, 2 and 3 (1.0 wt. %, 0.1 wt. %, and 0.01 wt. % idebenone) respectively produced a 44%, 33%, and 31% reduction in the number of sunburn cells when compared with normal, untreated skin. Thus, the application of idebenone and its derivatives containing compositions to skin prior to exposure to UV radiation reduces the formation of sunburn cells in human skin.

Referring now to Examples 3 and 4 below, it has also been found that the method of the invention provides a 100% reduction in the increase in the number of sunburn cells formed in skin treated with glycolic acid (neutralized to pH 3.8) and exposed to UV radiation.

### EXAMPLE 3

An Idebenone-containing Formula 4 and a Formula 5 (Placebo) were prepared as follows:

w/w %
Formula:

| | 4 | 5 |
|---|---|---|
| Idebenone | 0.5 | — |
| SD Alcohol 40B | 67.5 | 67.5 |
| DI Water | 22.0 | 22.5 |
| Ammonium Glycolate | 10.0 | 10.0 |
| and Glycolic Acid | | |
| (Neutralized to 3.8) | | |

To prepare Formula 4, the idebenone was added directly to the SD Alcohol 40B while stirring until dissolved. DI Water was then added to the solution with continued stirring followed by 10% Ammonium Glycolate and Glycolic Acid (Neutralized to 3.8). To prepare Formula 5, the same procedure was followed except that idebenone was not added to the composition.

### EXAMPLE 4

The idebenone-containing Formula 4 and the placebo Formula 5 of Example 3 were applied to human skin in a 2-week human sun burn cell assay study.

The panel composition was six different healthy adult volunteers between the ages of 18 and 53. All were in excellent health, and without any significant internal or dermatological diseases. The subjects were carefully screened to insure they were not taking any medications. All had a clear back free of blemishes or a tan and were of skin types II and III according to the following classification scheme:
Type I: always burns easily, never tans (sensitive)
Type II: always burns easily; tans minimally (sensitive)
Type III: burns moderately; tans gradually—normal skin (light brown)

The subjects were given detailed instructions to avoid any direct exposure to sunlight and to minimize any incidental exposure to their backs for the entire duration of the study. No other topical products were allowed, and no medications including over-the-counter (OTC) products were to be used except for Acetaminophen (e.g., common Tylenol®) for the relief of transient pains or headaches.

Individual test sites were delineated over the midback region. The test products were randomly allocated amongst the test sites according to a randomization schedule prepared by the investigator. The subjects came in once daily to the laboratory and received supervised applications of the test products to the allocated test sites. All test products were dispensed by a technician using 1 cc disposable plastic tuberculin syringes. Each site received 100 μl (2 μl/square cm) of, as appropriate, Formula 4 or 5 of Example 3, or neither formula (normal, untreated skin site). The test product was then spread uniformly throughout the 5×10 cm rectangular test site using a finger cot. The subjects received once daily application for two consecutive weeks (except on weekends).

The light source used was a 150 watt xenon arc solar simulator equipped with a UV reflecting diachronic mirror and a 1 mm thick Schott WG-320 filter to produce simulation of the solar spectrum. A 1 mm thick UG5 filter was added to remove reflected heat and remaining visible radiation. Warm up time of the lamp before use was 20-25 minutes. Total irradiance at skin level was measured with a calibrated Eppley Thermopile and the UVB component was monitored with a UVB radiometer (International Light Inc, Newburyport, Mass.). The size of the irradiated field was approximately a 1 cm diameter circle.

Two days prior to the end of the study, the MED (minimal erythema dose) for each subject was determined by exposing several normal untreated skin sites over the midback area to a series of exposures in 25% dose increments from the solar simulator. The MED was defined as the time of exposure required to produce a minimally perceptible erythema 20±4 hours after exposure. Visual grading of the MED was done under standardized lighting conditions when the subjects returned to the testing facility approximately 24 hours after irradiation. The MED was recorded in the appropriate case record form.

Approximately ten minutes after the last topical application of the test products, a circular area measuring 1 cm in diameter within each treated test site was exposed once to a signal does of 1.5 MED's. The MED was based on the determination in the nearby normal skin site using the solar stimulator. 20±2 hours later, shave biopsy (approximately 4×4 mm) was obtained from each irradiated site following injection of a local anesthetic (xylocaine). The skin specimens were immediately fixed in 10% buffered formalin.

Histology: the fixed specimens were processed routinely, embedded in paraffin and then sectioned and stained with hematoxylin-eosin. The numbers of sun burn cells were determined in at least 12 sections at 50 u intervals. A minimum of 70 High Power Fields (HPF) was counted from each biopsy and the average number of sun burn cells per HPF determined. All specimens were counted in a blinded manner by the investigator, where all slides were identified only by subject number and test code.

It has been found that the method of the invention prevented an increase in sun sensitivity, as demonstrated by prevention of increase in sunburn cell formation, normally caused by a product containing 10% glycolic acid when applied topically and exposed to the same degree of UV radiation.

The mean number of sunburn cells (SBC) for each subject is set forth below in Table II.

TABLE II
Mean Number of SBC Per High Power Field

| Subject | | | |
|---|---|---|---|
| Number | Site A | Site B | Site C |
| 1 | 4.86 | 9.28 | 3.16 |
| 2 | 1.03 | 3.18 | 0.75 |
| 3 | 0.78 | 1.22 | 2.18 |
| 4 | 0.13 | 0.21 | 0.36 |
| 5 | 2.17 | 1.32 | 1.68 |
| 6 | 3.33 | 3.06 | 4.24 |
| Mean | 2.05 | 3.50 | 2.06 |
| Delta % | 0.00 | −70.0 | N/A |

Site A: Formula 4 (0.5 wt. % idebenone + 10 wt. % glycolic acid)
Site B: Formula 5 (10 wt. % glycolic acid)
Site C: Normal untreated skin

Conclusion: Formula 4 (0.5 wt. % idebenone+10 wt. % glycolic acid) prevented the sun sensitivity produced by a 10 wt. % glycolic acid-containing composition when compared with normal, untreated skin. Thus, the application of Idebenone and/or its derivatives containing compositions to skin prior to exposure to UV radiation prevents the increase in formation of sunburn cells in human skin that can be produced by glycolic acid-containing compositions and the like.

While the invention has been described in connection with preferred embodiments, the description is not intended to limit the scope of the invention to a particular form set forth, but, on the contrary, it is intended to cover such alternatives, modifications and equivalents as may be included within the spirit and scope of the invention as defined by the appended claims.

1. A method for reducing the formation of sunburn cells in human skin, comprising:
applying to the skin a topical preparation comprising an amount of an agent effective to reduce the formation of sunburn cells in human skin, the agent comprising idebenone or a derivative of idebenone; and
exposing the skin to ultraviolet radiation.

2. The method as recited in claim 1 wherein the ultraviolet radiation includes ultraviolet B radiation from sunlight.

3. The method as recited in claim 1 wherein the ultraviolet radiation includes ultraviolet B radiation from a man-made ultraviolet radiation source.

4. The method as recited in claim 1 wherein the idebenone or derivative of idebenone has a concentration of from about 0.001% to about 30% by weight of the topical preparation.

5. The method as recited in claim 1 wherein the idebenone or derivative of idebenone has a concentration of from about 0.01% to about 10.0% by weight of the topical preparation.

6. The method as recited in claim 1 wherein the idebenone or derivative of idebenone has a concentration of from about 0.10% to about 2.0% by weight of the topical preparation.

7. The method as recited in claim 1 wherein the idebenone or derivative of idebenone has a concentration of from about 0.5% to about 1.0% by weight of the topical preparation.

8. The method as recited in claim 1 wherein the topical preparation includes at least one of an anhydrous preparation and an oil-free preparation.

9. The method as recited in claim 1 wherein the topical preparation includes at least one of an oil-in-water, a water-in-oil, and a water-in-oil-in-water emulsion.

10. The method as recited in claim 1 wherein the topical preparation is in the form of at least one of a lotion, a cream, a gel, a solution, a spray, a cleanser, a powder, an ointment, a wax, a lipstick, a soap, a shampoo, and a hydroalcoholic solution.

11. The method as recited in claim 1 wherein the applying is performed prior to the exposing.

12. The method as recited in claim 1 wherein, relative to untreated skin, there is a 1 to 100% reduction in the number of sunburn cells when the applying is performed prior to the exposing.

13. The method as recited in claim 1 wherein, relative to skin treated with an alpha hydroxy acid, a beta hydroxy acid, and/or a Retinoid, there is a 1 to 100% reduction in the number of sunburn cells when the applying is performed prior to the exposing.

14. The method as recited in claim 1 wherein the applying is performed at least once a day.

15. The method as recited in claim 1 wherein the applying is performed prior to engaging in outdoor activities.

16. The method as recited in claim 1 wherein the skin is facial skin.

17. The method as recited in claim 1 wherein the skin is skin on the neck, arms, or legs.

18. The method as recited in claim 1 wherein the topical preparation is a cosmetic preparation.

19. The method as recited in claim 1 wherein the topical preparation is a dermatological preparation.

20. The method as recited in claim 1 wherein the topical preparation is an over-the-counter preparation.

21. The method as recited in claim 1 wherein the topical preparation is a pharmaceutical preparation.

22. The method as recited in claim 1 wherein the topical preparation further comprises at least one antioxidant.

23. The method as recited in claim 1 wherein the topical preparation further comprises at least one antioxidant selected from the group consisting of Vitamin E, Vitamin C, Glutathione, Superoxide Dismutase, Catalase, Co-Enzyme Q10, Green Tea Extracts, alpha lipoic acid, and N-furfuryladenine.

24. The method as recited in claim 1 wherein the topical preparation further comprises at least one ultraviolet filter substance.

25. The method as recited in claim 1 wherein the topical preparation further comprises at least one ultraviolet filter substance selected from the group consisting of avenobenzene, cinoxate, dioxybenzone, homosalate, menthyl anthranilate, octocrylene, octyl methoxycinnamate, octyl salicylate, oxybenzone, padimate O, phenylbenzimidazole sulfonic acid, sulisobenzone, titanium dioxide, trolamine salicylate, and zinc oxide.

26. The method as recited in claim 1 wherein the derivative of idebenone is selected from the group consisting of a salt of idebenone, an ester of idebenone, and a protein-bound form of idebenone.

27. A method for preventing an increase in sun sensitivity in human skin in the presence of a compound capable of causing the increase in sun sensitivity, the method comprising:
applying to the skin a topical preparation comprising an amount of an agent effective to reduce the formation of sunburn cells in human skin, the agent comprising idebenone or a derivative of idebenone; and
exposing the skin to ultraviolet radiation.

28. The method as recited in claim 27 wherein the increase in sun sensitivity includes an increase, relative to untreated skin, in sunburn cell formation upon the exposing.

29. The method as recited in claim 27 wherein the compound is included in the topical preparation so that the compound and the agent are applied to the skin together.

30. The method as recited in claim 27 wherein the compound is selected from the group consisting of an alpha hydroxy acid, a beta hydroxy acid, and a retinoid.

31. The method as recited in claim 27 wherein the applying the topical preparation is performed prior to an applying of the compound to the skin.

32. The method as recited in claim 27 wherein the applying the topical preparation is performed after an applying of the compound to the skin.

33. The method as recited in claim 27 wherein the applying the topical preparation is performed concomitant with an applying of the compound to the skin.

34. The method as recited in claim 27 wherein the idebenone or derivative of idebenone has a concentration of from about 0.001% to about 30% by weight of the topical preparation.

35. The method as recited in claim 27 wherein the topical preparation is in the form of at least one of a lotion, a cream, a gel, a solution, a spray, a cleanser, a powder, an ointment, a wax, a lipstick, a soap, a shampoo, and a hydroalcoholic solution.

36. The method as recited in claim 27 wherein the applying the topical preparation is performed prior to the exposing.

37. The method as recited in claim 27 wherein, relative to untreated skin, there is a 1 to 100% reduction in the number of sunburn cells when the applying is performed prior to the exposing.

38. The method as recited in claim 27 wherein the derivative of idebenone is selected from the group consisting of a salt of idebenone, an ester of idebenone, and a protein-bound form of idebenone.

39. A method for reducing the formation of sunburn cells in human skin, comprising making available a topical preparation directed to reducing the formation of sunburn cells in human skin, the topical preparation comprising an amount of an agent effective to reduce the formation of sunburn cells in human skin, the agent comprising idebenone or a derivative of idebenone.

40. The method as recited in claim 39 wherein the idebenone or derivative of idebenone has a concentration of from about 0.001% to about 30% by weight of the topical preparation.

41. The method as recited in claim 39 wherein the topical preparation is in the form of at least one of a lotion, a cream, a gel, a solution, a spray, a cleanser, a powder, an ointment, a wax, a lipstick, a soap, a shampoo, and a hydroalcoholic solution.

42. The method as recited in claim 39 wherein the derivative of idebenone is selected from the group consisting of a salt of idebenone, an ester of idebenone, and a protein-bound form of idebenone.

43. The method as recited in claim 39 wherein the topical preparation further comprises at least one ultraviolet filter substance.

44. The method as recited in claim 39 wherein the topical preparation further comprises at least one ultraviolet filter substance selected from the group consisting of avenobenzene, cinoxate, dioxybenzone, homosalate, menthyl anthranilate, octocrylene, octyl methoxycinnamate, octyl salicylate, oxybenzone, padimate O, phenylbenzimidazole sulfonic acid, sulisobenzone, titanium dioxide, trolamine salicylate, and zinc oxide.

45. A topical preparation for reducing the formation of sunburn cells in human skin, comprising:
an ultraviolet filter substance; and
an amount of idebenone or a derivative of idebenone effective to reduce the formation of sunburn cells in human skin

46. The preparation as recited in claim 45 wherein the ultraviolet filter substance is selected from the group consisting of avenobenzene, cinoxate, dioxybenzone, homosalate, menthyl anthranilate, octocrylene, octyl methoxycinnamate, octyl salicylate, oxybenzone, padimate O, phenylbenzimidazole sulfonic acid, sulisobenzone, titanium dioxide, trolamine salicylate, and zinc oxide.

47. The preparation as recited in claim 45 wherein the formation of sunburn cells occurs upon an exposing of the skin to ultraviolet radiation.

48. The preparation as recited in claim 45 wherein the idebenone or derivative of idebenone has a concentration of from about 0.001% to about 30% by weight of the preparation.

49. The preparation as recited in claim 45 wherein the derivative of idebenone is selected from the group consisting of a salt of idebenone, an ester of idebenone, and a protein-bound form of idebenone.

50. The preparation as recited in claim 45 wherein the preparation is in the form of at least one of a lotion, a cream, a gel, a solution, a spray, a cleanser, a powder, an ointment, a wax, a lipstick, a soap, a shampoo, and a hydroalcoholic solution.

51. A topical preparation for reducing the formation of sunburn cells in human skin, comprising:
a compound capable of causing an increase in sun sensitivity in human skin; and
an amount of idebenone or a derivative of idebenone effective to reduce the formation of sunburn cells in the human skin.

52. The preparation as recited in claim 51 wherein the compound is selected from the group consisting of an alpha hydroxy acid, a beta hydroxy acid, and a retinoid.

53. The preparation as recited in claim 51 wherein the increase in sun sensitivity includes an increase, relative to untreated skin, in sunburn cell formation upon an exposing of the skin to ultraviolet radiation.

54. The preparation as recited in claim 51 wherein the idebenone or derivative of idebenone has a concentration of from about 0.001% to about 30% by weight of the preparation.

55. The preparation as recited in claim 51 wherein the preparation is in the form of at least one of a lotion, a cream, a gel, a solution, a spray, a cleanser, a powder, an ointment, a wax, a lipstick, a soap, a shampoo, and a hydroalcoholic solution.

56. The preparation as recited in claim 51 wherein the derivative of idebenone is selected from the group consisting of a salt of idebenone, an ester of idebenone, and a protein-bound form of idebenone.
